(19) United States
(12) Patent Application Publication
(10) Pub. No.: US 2013/0281466 A1
(43) Pub. Date: Oct. 24, 2013

(54) ARTHROPOD PEST CONTROL COMPOSITION AND METHOD FOR CONTROLLING ARTHROPOD PESTS

(71) Applicants: Norihisa Sakamoto, Kasai-shi (JP); Emiko Sakamoto, Takarazuka-shi (JP); Atsushi Iwata, Tokyo (JP)

(72) Inventors: Norihisa Sakamoto, Kasai-shi (JP); Emiko Sakamoto, Takarazuka-shi (JP); Atsushi Iwata, Tokyo (JP)

(73) Assignee: SUMITOMO CHEMICAL COMPANY, LIMITED, Chuo-ku, Tokyo (JP)

(21) Appl. No.: 13/997,322

(22) PCT Filed: Feb. 18, 2011
(86) PCT No.: PCT/JP2011/054218

§ 371 Date: Jun. 24, 2013

(30) Foreign Application Priority Data
Dec. 27, 2010 (JP) 2010-289612

(51) Int. Cl. A01N 43/90; C07D 513/04; A01N 37/18; A01N 43/56; A01N 37/10; C07D 471/04; A01N 43/54

(52) U.S. Cl. CPC A01N 43/90; C07D 471/04; C07D 513/04; A01N 43/54; A01N 43/56; A01N 37/10; A01N 37/18
USPC 514/259.2; 514/259.41

(57) ABSTRACT

Disclosed is an arthropod pest control composition having an excellent controlling effect on arthropod pests, which comprises a compound represented by formula (I):

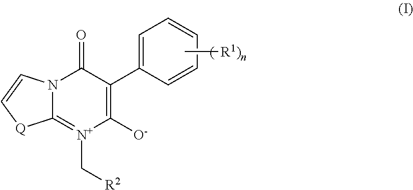

wherein each symbol is as defined in the description; and at least one disinfectant compound selected from Group (A).
Group (A): a group consisting of azoxystrobin, fluoxastrobin, pyraclostrobin, a compound represented by the following formula (1), trifloxystrobin, kresoxim-methyl, metominostrobin, picoxystrobin, enestrobin, dimoxystrobin, famoxadone, fenamidone and pyribencarb;

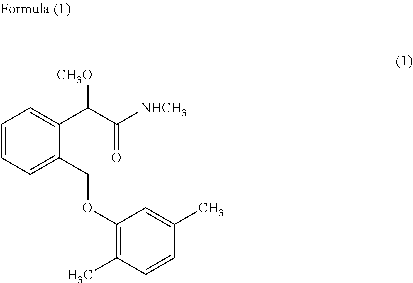

### TECHNICAL FIELD

The present application was filed claiming the priority of the Japanese Patent Application No. 2010-289612, the entire contents of which are herein incorporated by the reference.

The present invention relates to an arthropod pest control composition and a method for controlling arthropod pests.

### BACKGROUND ART

Heretofore, various compounds are known as active ingredients in arthropod pest control compositions (see, for example, Patent Literature 1 and Non-Patent Literature

### CITATION LIST

### Patent Literature

Patent Literature 1: WO 2009/099929

### Non-Patent Literature

Non-Patent Literature 1: The Pesticide Manual-15th edition (published BCPC); ISBN 978-1-901396-18-8

### SUMMARY OF INVENTION

### Technical Problem

An object of the present invention is to provide an arthropod pest control composition having an excellent control effect on arthropod pests.

### Solution to Problem

The present inventors have intensively studied for providing an arthropod pest control composition having an excellent control effect on arthropod pests, and finally found that a composition comprising a compound represented by formula (I) as described below, and a disinfectant compound selected from Group (A) as described below has an excellent control effect on arthropod pests, thereby attaining the present invention.

The present invention provides:

[1] An arthropod pest control composition comprising a compound represented by formula (I):

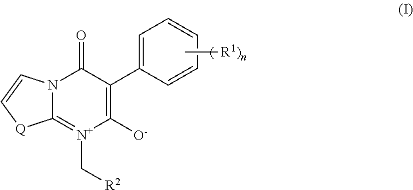

wherein,

Q represents CR⁵═CR⁶, S, O or NCH₃,

R¹ represents a halogen atom, cyano, nitro, an optionally halogenated C1-C4 alkyl group, an optionally halogenated C2-C4 alkenyl group, an optionally halogenated C2-C4 alkynyl group or an optionally halogenated C1-C4 alkoxy group,

n represents an integer of 0 to 3,

R² represents the following R^{2a}, R^{2b}, R^{2c} or R^{2d}:

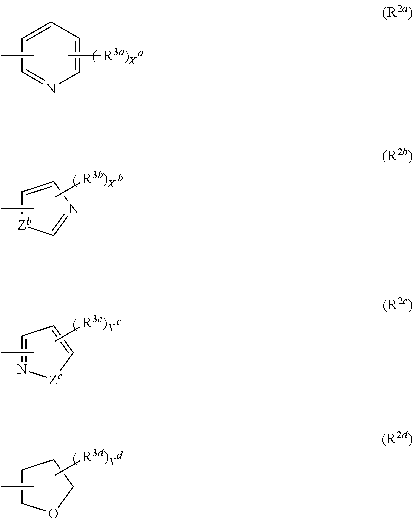

wherein,

R^{3a}, R^{3b} and R^{3c} each independently represent a halogen atom, cyano, nitro, an optionally halogenated C1-C4 alkyl group, an optionally halogenated C2-C4 alkynyl group or an optionally halogenated C1-C4 alkoxy group,

R^{3d} represents a halogen atom, cyano, nitro, an optionally halogenated C1-C4 alkyl group or an optionally halogenated C1-C4 alkoxy group,

X^{a}, X^{b}, X^{c} and X^{d} each independently represent 0, 1 or 2,

Z^{b} and Z^{c} each independently represent O, S or NR⁷,

R⁷ represents a hydrogen atom or an optionally halogenated C1-C4 alkyl group,

wherein,

when X^{a} represents 2, then two R^{3a}'s may be the same or different,

when X^{b} represents 2, then two R^{3b}'s may be the same or different,

when X^{c} represents 2, then two R^{3c}'s may be the same or different, and

when X^{d} represents 2, then two R^{3d}'s may be the same or different,

R⁵ represents a hydrogen atom or a fluorine atom, and

R⁶ represents a hydrogen atom, a fluorine atom, a difluoromethyl group or a trifluoromethyl group,

wherein,

when n represents 2 or 3, then plural R¹'s may be the same or different; and

at least one disinfectant compound selected from Group (A);
Group (A): a group consisting of azoxystrobin, fluoxastrobin, pyraclostrobin, a compound represented by the following formula (1), trifloxystrobin, kresoxim-methyl, metominostrobin, picoxystrobin, enestrobin, dimoxystrobin, famoxadone, fenamidone and pyribencarb;

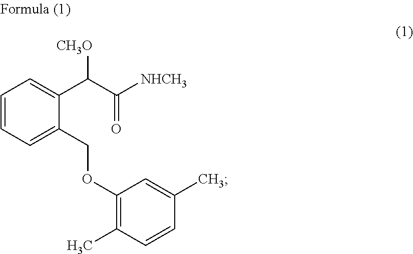

[2] The arthropod pest control composition according to the above [1], wherein the weight ratio of the compound represented by formula (I) to the disinfectant compound 10,000:1 to 0.01:1;

[3] The arthropod pest control composition according to the above [1] or [2], wherein the disinfectant compound is azoxystrobin, pyraclostrobin, a compound represented by the following formula (1) or trifloxystrobin;

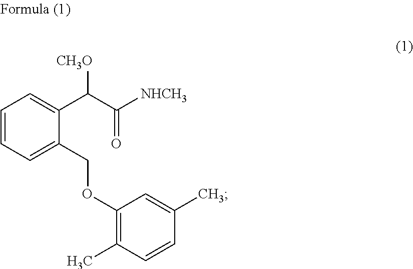

[4] The arthropod pest control composition according to any of the above [1] to [3], wherein the composition further comprises metalaxyl or metalaxyl M;
[5] The arthropod pest control composition according to the above [4], wherein the weight ratio of the compound represented by formula (I) to metalaxyl or metalaxyl M is 10,000:1 to 0.01:1;
[6] A method for controlling arthropod pests, which comprises applying an effective amount of the arthropod pest control composition according to any of the above [1] to [5] to plants or area in which plants are grown;
[7] The method for controlling arthropod pests according to the above [6], wherein the plants or area in which plants are grown is the seeds of plants.

### Effects of Invention

According to the present invention, it is possible to control arthropod pests.

The arthropod pest control composition of the present invention comprises a compound represented by the following formula (I) (hereinafter referred to as “the mesoionic compound”):

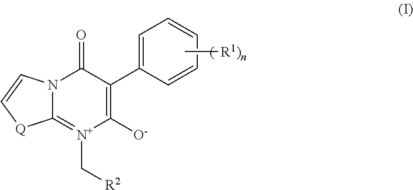

wherein,

Q represents CR⁵═CR⁶, S, O or NCH₃,

R¹ represents a halogen atom, cyano, nitro, an optionally halogenated C1-C4 alkyl group, an optionally halogenated C2-C4 alkenyl group, an optionally halogenated C2-C4 alkynyl group or an optionally halogenated C1-C4 alkoxy group,

n represents an integer of 0 to 3,

R² represents the following R^{2a}, R^{2b}, R^{2c} or R^{2d}:

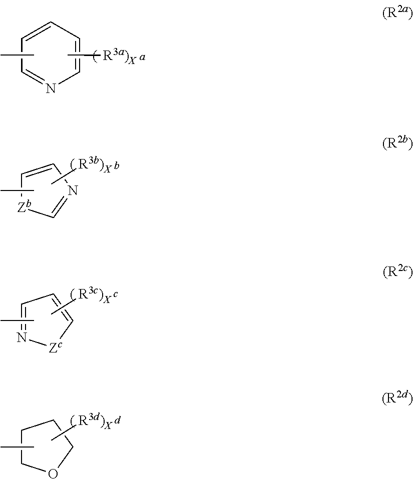

wherein,

R^{3a}, R^{3b} and R^{3c} each independently represent a halogen atom, cyano, nitro, an optionally halogenated C1-C4 alkyl group, an optionally halogenated C2-C4 alkynyl group or an optionally halogenated C1-C4 alkoxy group,

R^{3d} represents a halogen atom, cyano, nitro, an optionally halogenated C1-C4 alkyl group or an optionally halogenated C1-C4 alkoxy group,

X^{a}, X^{b}, X^{c} and X^{d} each independently represent 0, 1 or 2,

Z^{b} and Z^{c} each independently represent O, S or NR⁷,

R⁷ represents a hydrogen atom or an optionally halogenated C1-C4 alkyl group,

wherein,

when X^{a} represents 2, then two R^{3a}'s may be the same or different,

when X^{b} represents 2, then two R^{3b}'s may be the same or different,

when X^{c} represents 2, then two R^{3c}'s may be the same or different, and

when X^{d} represents 2, then two R^{3d}'s may be the same or different,

R⁵ represents a hydrogen atom or a fluorine atom, and

R⁶ represents a hydrogen atom, a fluorine atom, a difluoromethyl group or a trifluoromethyl group,

wherein,

when n represents 2 or 3, then plural R¹'s may be the same or different; and

at least one disinfectant compound selected from Group (A) (hereinafter referred to as “the disinfectant compound”);
Group (A): a group consisting of azoxystrobin, fluoxastrobin, pyraclostrobin, a compound represented by the following formula (1), trifloxystrobin, kresoxim-methyl, metominostrobin, picoxystrobin, enestrobin, dimoxystrobin, famoxadone, fenamidone and pyribencarb;

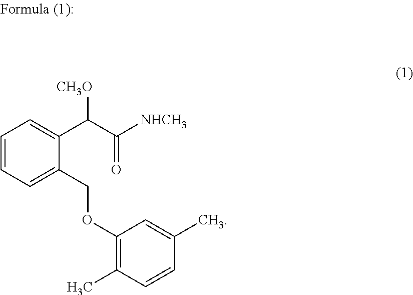

Examples of R¹, R^{2a}, R^{2b}, R^{2c}, R^{2d}, R^{3a}, R^{3b}, R^{3c}, R^{3d} and R⁷ in the formula (I) include as follows:

Examples of the “halogen” represented by R¹, R^{3a}, R^{3b}, R^{3c} or R^{3d} include a fluorine atom, a chlorine atom, a bromine atom and an iodine atom.

Examples of the “optionally halogenated C1-C4 alkyl group” represented by R¹, R^{3a}, R^{3b}, R^{3c}, R^{3d} or R⁷ include a methyl group, a trifluoromethyl group, a trichloromethyl group, a chloromethyl group, a dichloromethyl group, a fluoromethyl group, a difluoromethyl group, an ethyl group, a pentafluoroethyl group, a 2,2,2-trifluoroethyl group, a 2,2,2-trichloroethyl group, a propyl group, a 1-methylethyl group, a 1-trifluoromethyltetrafluoroethyl group, a butyl group, a 2-methylpropyl group, a 1-methylpropyl group and a 1,1-dimethylethyl.

Examples of the “optionally halogenated C2-C4 alkenyl group” represented by R¹ include a 2-propenyl group, a 3-chloro-2-propenyl group, a 2-chloro-2-propenyl group, a 3,3-dichloro-2-propenyl group, a 2-butenyl group, a 3-butenyl group and a 2-methyl-2-propenyl group.

Examples of the “optionally halogenated C1-C4 alkynyl group” represented by R¹, R^{3a}, R^{3b} or R^{3c} include a 2-propynyl group, a 3-chloro-2-propynyl group, a 3-bromo-2-propynyl group, a 2-butynyl group and a 3-butynyl group.

Examples of the “optionally halogenated C1-C4 alkoxy group” represented by R¹, R^{3a}, R^{3b}, R^{3c} or R^{3d} include a methoxy group, a trifluoromethoxy group, an ethoxy group, a 2,2,2-trifluoroethoxy group, a propoxy group, a 1-methylethoxy group, a butoxy group, a 2-methylpropoxy group, a 1-methylpropoxy group and a 1,1-dimethylethoxy group.

Examples of R^{2a} include a 6-fluoro-3-pyridyl group, a 6-chloro-3-pyridyl group, a 6-bromo-3-pyridyl group, a 6-methyl-3-pyridyl group, a 6-cyano-3-pyridyl group, a 3-pyridyl group, a 2-pyridyl group, and a 5,6-dichloro-3-pyridyl group.

Examples of R^{2b} include a 2-fluoro-5-thiazolyl group, a 2-chloro-5-thiazolyl group, a 2-bromo-5-thiazolyl group, a 2-methyl-5-thiazolyl group, a 5-thiazolyl group, a 2-fluoro-5-oxazolyl group, a 2-chloro-5-oxazolyl group, a 5-oxazolyl group, a 2-chloro-1-methyl-5-imidazolyl group and a 2-fluoro-1-methyl-5-imidazolyl group.

Examples of R^{2c} include a 1-methyl-4-pyrazolyl group and a 3-methyl-5-isoxazolyl group.

Examples of R^{2d} include a tetrahydrofuran-2-yl group and a tetrahydrofuran-3-yl group.

The compound represented by formula (I) wherein Q represents CR⁵═CR⁶ is represented by the following formula (II-a):

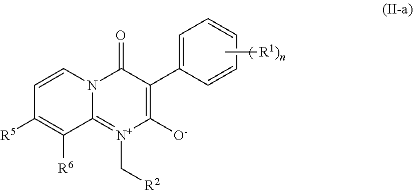

wherein R¹, R², R⁵, R⁶ and n are as defined above.

The compound represented by formula (I) wherein Q represents S is represented by the following formula (II-b):

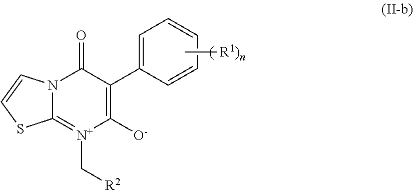

wherein R¹, R² and n are as defined above.

The compound represented by formula (I) wherein Q represents O is represented by the following formula (II-c):

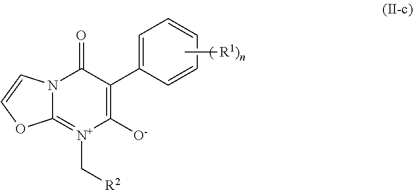

wherein R¹, R² and n are as defined above.

The compound represented by formula (I) wherein Q represents NCH₃ is represented by the following formula (II-d):

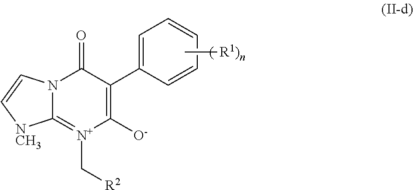

wherein R¹, R² and n are as defined above.

Examples of the mesoionic compound include as follows:

those represented by formula (I) wherein n represents 0 or 1, when n represents 0, R² represents a 2-chloro-5-thiazolyl group, a 1-methyl-4-pyrazolyl group, a 6-chloro-3-pyridyl group or a tetrahydrofuran-3-yl group, and Q is CH═CH or S, and when n represents 1, R² represents a fluorine atom, a chlorine atom, a bromine atom, a methyl group, a methoxy group, a trifluoromethyl group or a trifluoromethoxy group, and R² represents a 2-chloro-5-thiazolyl group, a 6-chloro-3-pyridyl group, a 1-methyl-4-pyrazolyl group or a tetrahydrofuran-3-yl group, and Q is CH═CH or S;
those represented by formula (I) wherein n represents 0 or 1, and when n represents 1, then R¹ represents a fluorine atom, a chlorine atom, a bromine atom, a methyl group, a methoxy group, a trifluoromethyl group or a trifluoromethoxy group, and R² represents a 2-chloro-5-thiazolyl group, a 6-chloro-3-pyridyl group, a 1-methyl-4-pyrazolyl group or a tetrahydrofuran-3-yl group, and Q is CH═CH or S;
those represented by formula (I) wherein n represents 0 or 1, when n represents 0, R² represents a 2-chloro-5-thiazolyl group, and Q represents CH═CH, and when n represents 1, R¹ represents a fluorine atom, a trifluoromethyl group or a trifluoromethoxy group, and R² represents a 2-chloro-5-thiazolyl group or a 6-chloro-3-pyridyl group, and Q represents CH═CH;
those represented by formula (I) wherein n represents 0 or 1, and when n represents 1, R¹ represents a fluorine atom, a trifluoromethyl group or a trifluoromethoxy group, and R² represents a 2-chloro-5-thiazolyl group or a 6-chloro-3-pyridyl group, and Q represents CH═CH.

Specific examples of the mesoionic compound include those represented by formula (I-a):

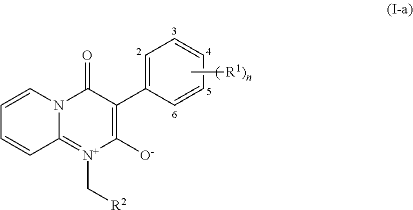

wherein a combination of n, R¹ and R² represents any combination as shown in Tables 1 and 2.

TABLE 1

| | | | |
|---|---|---|---|
| Compound | | | |
| No. | n | R¹ | R² |

| | | | | |
|---|---|---|---|---|
| 1 | 0 | — | — | 2-chloro-5-thiazolyl |
| 2 | 0 | — | — | 1-methyl-4-pyrazolyl |
| 3 | 0 | — | — | tetrahydrofuran-3-yl |
| 4 | 1 | 2-F | — | 2-chloro-5-thiazolyl |
| 5 | 1 | 2-F | — | 6-chloro-3-pyridyl |
| 6 | 1 | 2-F | — | 1-methyl-4-pyrazolyl |
| 7 | 1 | 2-OCF₃ | — | 2-chloro-5-thiazolyl |
| 8 | 1 | 2-OCF₃ | — | 6-chloro-3-pyridyl |
| 9 | 1 | 3-Br | — | 6-chloro-3-pyridyl |
| 10 | 1 | 3-Br | — | 2-chloro-5-thiazolyl |
| 11 | 1 | 3-CF₃ | — | 2-chloro-5-thiazolyl |
| 12 | 1 | 3-CF₃ | — | 6-chloro-3-pyridyl |
| 13 | 1 | 3-F | — | 6-chloro-3-pyridyl |
| 14 | 1 | 3-F | — | 2-chloro-5-thiazolyl |
| 15 | 1 | 3-OCF₃ | — | 2-chloro-5-thiazolyl |
| 16 | 1 | 3-OCF₃ | — | 6-chloro-3-pyridyl |
| 17 | 1 | 3-OCF₃ | — | 1-methyl-4-pyrazolyl |
| 18 | 1 | 4-CF₃ | — | 6-chloro-3-pyridyl |
| 19 | 1 | 4-CF₃ | — | 2-chloro-5-thiazolyl |
| 20 | 1 | 4-F | — | 2-chloro-5-thiazolyl |

TABLE 2

| Compound No. | n | R¹ | | R² |
|---|---|---|---|---|
| 21 | 1 | 4-F | — | 1-methyl-4-pyrazolyl |
| 22 | 1 | 4-F | — | tetrahydrofuran-3-yl |
| 23 | 1 | 4-OCF₃ | — | 6-chloro-3-pyridyl |
| 24 | 1 | 4-OCF₃ | — | 2-chloro-5-thiazolyl |
| 25 | 2 | 2-F | 4-F | 2-chloro-5-thiazolyl |
| 26 | 2 | 2-F | 4-F | 1-methyl-4-pyrazolyl |
| 27 | 2 | 2-F | 4-F | tetrahydrofuran-3-yl |
| 28 | 2 | 2-F | 5-CF₃ | 2-chloro-5-thiazolyl |
| 29 | 2 | 2-F | 5-CF₃ | 1-methyl-4-pyrazolyl |
| 30 | 2 | 2-F | 6-F | 2-chloro-5-thiazolyl |
| 31 | 2 | 2-F | 6-F | 6-chloro-3-pyridyl |
| 32 | 2 | 3-CF₃ | 4-F | 6-chloro-3-pyridyl |
| 33 | 2 | 3-CF₃ | 4-F | 2-chloro-5-thiazolyl |
| 34 | 2 | 3-F | 4-F | 6-chloro-3-pyridyl |
| 35 | 2 | 3-F | 4-F | 2-chloro-5-thiazolyl |
| 36 | 2 | 3-CF₃ | 5-CF₃ | 2-chloro-5-thiazolyl |
| 37 | 2 | 3-CF₃ | 5-CF₃ | 6-chloro-3-pyridyl |
| 38 | 2 | 3-F | 5-F | 6-chloro-3-pyridyl |
| 39 | 2 | 3-F | 5-F | 2-chloro-5-thiazolyl |

In Tables 1 and 2, the “3-” as shown in the substituents “3-OCF₃”, “3-Br”, etc. of R¹ means that such substituent is present as R¹ at the 3-position of the benzene ring in the above formula (I-a).

Compounds represented by formula (I-b):

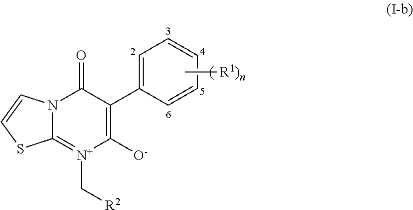

wherein a combination of n, R¹ and R² represents any combination as shown in Table 3.

TABLE 3

| Compound No. | n | R¹ | | R² |
|---|---|---|---|---|
| 40 | 0 | — | — | 6-chloro-3-pyridyl |
| 41 | 0 | — | — | 2-chloro-5-thiazolyl |
| 42 | 1 | 2-F | — | 6-chloro-3-pyridyl |
| 43 | 1 | 2-F | — | 2-chloro-5-thiazolyl |
| 44 | 1 | 3-OCF₃ | — | 6-chloro-3-pyridyl |
| 45 | 1 | 3-OCF₃ | — | 2-chloro-5-thiazolyl |
| 46 | 1 | 4-F | — | 6-chloro-3-pyridyl |
| 47 | 1 | 4-F | — | 2-chloro-5-thiazolyl |
| 48 | 2 | 2-F | 3-F | 6-chloro-3-pyridyl |
| 49 | 2 | 2-F | 3-F | 2-chloro-5-thiazolyl |
| 50 | 2 | 2-F | 4-F | 2-chloro-5-thiazolyl |
| 51 | 2 | 2-F | 4-F | 6-chloro-3-pyridyl |
| 52 | 2 | 3-OCF₃ | 5-Br | 2-chloro-5-thiazolyl |
| 53 | 2 | 3-OCF₃ | 5-Br | 6-chloro-3-pyridyl |

In Table 3, the “3-” as shown in the substituents “3-OCF₃”, “3-F”, etc. of R¹ means that such substituent is present as R¹ at the 3-position of the benzene ring in the above formula (I-b).

The mesoionic compound to be used in the present invention includes the forms represented by formula (I) and its ionized forms represented by the formula different from formula (I), and can be used in any of the above forms alone or in a combination of two or more thereof.

The mesoionic compound can be prepared, for example, by a process described in WO 2009/099929.

Azoxystrobin, fluoxastrobin, pyraclostrobin, trifloxystrobin, kresoxim-methyl, metominostrobin, picoxystrobin, enestrobin, dimoxystrobin, famoxadone, fenamidone, metalaxyl and metalaxyl M to be used in the present invention are known as described, for example, at pages 62, 538, 971, 1167, 688, 783, 910, 1068, 383, 458, 462, 737 and 739 of “The Pesticide Manual-15th edition (published BCPC); ISBN 978-1-901396-18-8”. These compounds are commercially available, or can be produced by known methods.

Pyribencarb to be used in the present invention is known and can be prepared, for example, by a process described in WO 2001/010825.

The compound represented by the following formula (1):

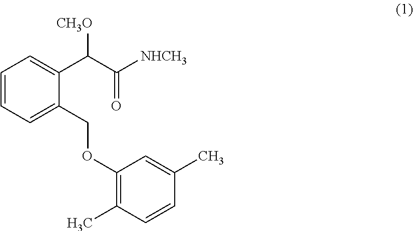

(hereinafter referred to as “the active compound (1)”) to be used in the present invention is disclosed in, for example, WO 1995/27693, and can be prepared, for example, by a process described in WO 1995/27693.

The active compound (1) has an asymmetric carbon atom, and so there are two enantiomers of the compound, the R- and S-forms, based on the asymmetric carbon atom. The R-form is represented by the following formula (1a):

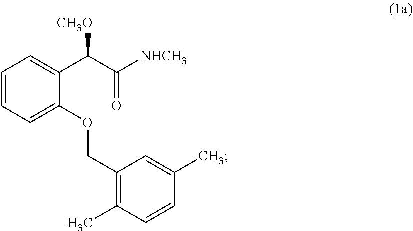

and the S-form is represented by the following formula (1b):

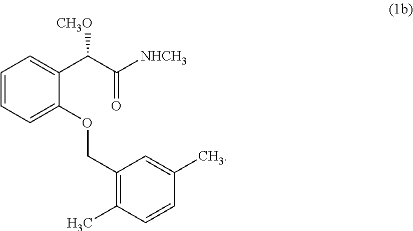

The active compound (1) may be used in any ratio of enantiomers in the present invention.

Azoxystrobin, fluoxastrobin, pyraclostrobin, trifloxystrobin, kresoxim-methyl, metominostrobin, picoxystrobin, enestrobin, dimoxystrobin, famoxadone, fenamidone, pyribencarb and the active compound (1) to be used in the present invention are disinfectant compounds known to show antimicrobial activity due to the inhibition of respiration, caused by the inhibitory action of the compounds on mitochondrial electron transport systems (Complex III) in pests.

The disinfectant compounds of the arthropod pest control composition are preferably azoxystrobin, pyraclostrobin, active compound (1), and trifloxystrobin.

In the arthropod pest control composition of the present invention, the weight ratio of the disinfectant compound to the mesoionic compound is not particularly limited, but is generally 0.001 to 100,000 parts by weight, preferably 0.01 to 10,000 parts by weight of the disinfectant compound, relative to 100 parts by weight of the mesoionic compound, i.e., [the compound represented by formula (I)]:(disinfectant compound)=10,000:1 to 0.01:1, more preferably [the compound represented by formula (I)]:(disinfectant compound)=1,000:1 to 0.1:1.

The arthropod pest control composition of the present invention may further contain, in addition to the mesoionic compound and the disinfectant compound, other agricultural active compounds. Examples of other agricultural active compounds include metalaxyl and metalaxyl M, preferably metalaxyl M.

When the arthropod pest control composition of the present invention further contains metalaxyl or metalaxyl M in addition to the mesoionic compound and the disinfectant compound, the weight ratio of metalaxyl or metalaxyl M to the mesoionic compound is not particularly limited, but is generally 0.001 to 100,000 parts by weight, preferably 0.01 to 10,000 parts by weight of metalaxyl or metalaxyl M, relative to 100 parts by weight of the mesoionic compound, i.e., [the compound represented by formula (I)]:(metalaxyl or metalaxyl M)=10,000:1 to 0.01:1, more preferably [the compound represented by formula (I)]:(metalaxyl or metalaxyl M)=1,000:1 to 0.1:1.

The arthropod pest control composition of the present invention may be prepared by simply mixing the mesoionic compound with the disinfectant compound, but generally by mixing the mesoionic compound, the disinfectant compound and an inert carrier, and if necessary, a surfactant and/or other formulation additives, and then formulating the mixture into a dosage form such as an oil solution, an emulsifiable concentrate, a suspension concentrate, a wettable powder, a water dispersible granule, a dust, or a granule.

Thus formulated arthropod pest control composition may be used directly, or after the addition of other inert ingredients, as an arthropod pest control agent.

The total amount of the mesoionic compound and the disinfectant compound in the arthropod pest control composition of the present invention is generally 0.1 to 99% by weight, preferably 0.2 to 90% by weight, more preferably 1 to 80% by weight.

Examples of the solid carrier used for formulation of the arthropod pest control composition include fine powders or granules of minerals (e.g., kaolin clay, attapulgite clay, bentonite, montmorillonite, acidic white clay, pyrophylite, talc, diatomaceous earth, and calicite), natural organic substances (e.g., corncob flour, and walnut shell flour), synthetic organic substances (e.g., urea), salts (e.g., calcium carbonate, and ammonium sulfate), and synthetic inorganic substances (e.g., synthetic hydrated silicon oxide).

Examples of the liquid carrier include aromatic hydrocarbons (e.g., xylene, alkylbenzene, and methyl naphthalene), alcohols (e.g., 2-propanol, ethylene glycol, propylene glycol, and ethylene glycol monoethyl ether), ketones (e.g., acetone, cyclohexanone, and isophorone), vegetable oils (e.g., soybean oil, and cotton oil), petroleum-based aliphatic hydrocarbons, esters, dimethylsulfoxide, acetonitrile, and water.

Examples of the surfactant include anionic surfactants (e.g., alkyl sulfate ester salts, alkylaryl sulfonates, dialkyl sulfosuccinates, polyoxyethyle alkylaryl ether phosphate ester salts, ligninsulfonates, and naphthalene sulfonate formaldehyde polycondensates), nonionic surfactants (e.g., polyoxyethylene alkylaryl ethers, polyoxyethylene alkylpolyoxypropylene block copolymers, and sorbitan fatty acid esters), and cationic surfactants (e.g., alkyl trimethyl ammonium salts).

Examples of the formulation additives include water-soluble polymers (e.g., polyvinyl alcohol, and polyvinyl pyrrolidone), polysaccharides [e.g., gum arabic, alginic acid and a salt thereof, CMC (carboxymethyl cellulose), and xanthane gum], inorganic substances (e.g., aluminum magnesium silicate, and alumina-sol), preservatives, colorants, and stabilizers [e.g. PAP (isopropyl acid phosphate), and BHT].

The arthropod pest control composition of the present invention can be used for protecting plants from damage due to eating or sucking by arthropod pests.

Examples of arthropod pests on which the arthropod pest control composition of the present invention has controlling effect include as described below:

Hemiptera:

Delphacidae such as Laodelphax striatellus, Nilaparvata lugens, Sogatella furcifera; Deltocephalidae such as Nephotettix cincticeps, Nephotettix virescens, Recilia dorsalis, Empoasca onukii; Aphididae such as Aphis gossypii, Myzus persicae, Brevicoryne brassicae, Aphis spiraecola, Macrosiphum euphorbiae, Aulacorthum solani, Rhopalosiphum padi, Toxoptera citricidus, Hyalopterus pruni, Eriosoma lanigerum; Pentatomidae such as Nezara antennata, Trigonotylus caelestialium, Graphosoma rubrolineatum, Eysarcoris lewisi, Riptortus clavetus, Leptocorisa chinensis, Eysarcoris parvus, Halyomorpha mista, Nezara viridula, and Lygus lineolaris; Aleyrodidae such as Trialeurodes vaporariorum, Bemisia tabaci, Dialeurodes citri, and Aleurocanthus spiniferus; Coccoidea such as Aonidiella aurantii, Comstockaspis perniciosa, Unaspis citri, Ceroplastes rubens, Icerya purchasi, Planococcus kraunhiae, Pseudococcus longispinis, and Pseudaulacaspis pentagona; Tingidae; Cimicoidea such as Cimex lectularius; Psyllidae such as Cacopsylla pyricola; etc.

Lepidoptera:

Pyralidae such as Chilo suppressalis, Tryporyza incertulas, Cnaphalocrocis medinalis, Notarcha derogata, Plodia interpunctella, Ostrinia furnacalis, Hellula undalis, and Pediasia teterrellus; Noctuidae such as Spodoptera litura, Spodoptera exigua, Pseudaletia separata, Sesamia inferens, Mamestra brassicae, Agrotis Ipsilon, Plusia nigrisigna, Trichoplusia ni, Thoricoplusia spp., Heliothis spp., and Helicoverpa spp.; Pieridae such as Pieris rapae; Tortricidae such as Adoxophyes spp., Grapholita molesta, Leguminivora glycinivorella, Matsumuraeses azukivora, Adoxophyes orana fasciata, Adoxophyes honmai., Homona magnanima, Archips fuscocupreanus, and Cydia pomonella; Gracillariidae such as Caloptilia theivora, and Phyllonorycter ringoneella; Carposimidae such as Carposina niponensis; Lyonetiidae such as Lyonetia spp.; Lymantriidae such as Lymantria spp., and Euproctis spp.; Yponomeutidae such as Plutella xylostella; Gelechiidae such as Pectinophora gossypiella, and Phthorimaea operculella; Arctiidae such as Hyphantria cunea; Tineidae such as Tinea translucens, and Tineola bisselliella; Tuta absoluta; etc.

Thysanoptera:

Thripidae such as Frankliniella occidentalis, Thrips parmi, Scirtothrips dorsalis, Thrips tabaci, Frankliniella intonsa, Frankliniella fusca, Stenchaetothrips biformis, Haplothrips aculeatus; etc.

Diptera:

Agromyzidae such as Hylemya antiqua, Hylemya platura, Agromyza oryzae, Hydrellia griseola, Chlorops oryzae, and Liriomyza trifolii; Dacus cucurbitae, Ceratitis capitata; etc.

Coleoptera:

Epilachna vigintioctopunctata, Aulacophora femoralis, Phyllotreta striolata, Oulema oryzae, Echinocnemus squameus, Lissorhoptrus oryzophilus, Anthonomus grandis, Callosobruchus chinensis, Sphenophorus venatus, Popillia japonica, Anomala cuprea, Diabrotica spp., Leptinotarsa decemlineata, Agriotes spp., Lasioderma serricorne; etc.

Orthoptera:

Gryllotalpa africana, Oxya yezoensis, Oxya japonica; etc.

Among the above arthropod pests, Delphacidae, Deltocephalidae, Aphididae, etc. are suitable for the present invention.

The arthropod pest control composition of the present invention may be used for controlling plant diseases, for example, diseases caused by Rhizoctonia spp. or Pythium spp. or Fusarium spp. in corn, rice, soybean, cotton, rapeseed, or wheat.

The arthropod pest control composition of the present invention can be used in agricultural lands such as fields, paddy fields, dry fields, lawns, and orchards or nonagricultural lands. The arthropod pest control composition of the present invention can be also used for controlling pests in agricultural lands, etc. in which “plants”, etc. are grown.

Examples of plants to which the arthropod pest control composition of the present invention can be applied include as described below:

Crops: corn, rice, wheat, barley, rye, oat, sorghum, cotton, soybean, peanut, buckwheat, sugar beet, rapeseed, sunflower, sugar cane, tobacco, etc.;

Vegetables: Solanaceae vegetables (eggplant, tomato, green pepper, hot pepper, potato, etc.), Cucurbitaceae vegetables (cucumber, pumpkin, zucchini, watermelon, melon, etc.), Cruciferae vegetables (Japanese radish, turnip, horseradish, kohlrabi, Chinese cabbage, cabbage, brown mustard, broccoli, cauliflower, rape, etc.), Compositae vegetables (burdock, garland chrysanthemum, artichoke, lettuce, etc.), Liliaceae vegetables (Welsh onion, onion, garlic, asparagus, etc.), Umbelliferae vegetables (carrot, parsley, celery, parsnip, etc.), Chenopodiaceae vegetables (spinach, Swiss chard, etc.), Labiatae vegetables (Japanese basil, mint, basil, etc.), strawberry, sweat potato, yam, aroid, etc.;

Fruit trees: pomaceous fruits (apple, common pear, Japanese pear, Chinese quince, quince, etc.), stone fleshy fruits (peach, plum, nectarine, Japanese plum, cherry, apricot, prune, etc.), citrus plants (Satsuma mandarin, orange, lemon, lime, grapefruit, etc.), nuts (chestnut, walnut, hazel nut, almond, pistachio, cashew nut, macadamia nut, etc.), berry fruits (blueberry, cranberry, blackberry, raspberry, etc.), grape, persimmon, olive, loquat, banana, coffee, date, coconut, oil palm, etc.;

Trees other than fruit trees: tea, mulberry, flowering trees (azalea, camellia, hydrangea, sasanqua, Japanese star anise, cherry, tulip tree, crape myrtle, orange osmanthus, etc.), street trees (ash tree, birch, dogwood, eucalyptus, ginkgo, lilac, maple tree, oak, poplar, cercis, Chinese sweet gum, plane tree, zelkova, Japanese arborvitae, fir tree, Japanese hemlock, needle juniper, pine, spruce, yew, spruce, elm, horse chestnut, etc.), coral tree, podocarpus, cedar, Japanese cypress, croton, Euonymus japonicus, Photinia glabra, etc.;

lawns: Zoysia (zoysiagrass, Zoysia matrella, etc.), Bermuda grasses (Cynodon dactylon, etc.), bent grasses (Agrostis alba, creeping bent grass, hiland bent, etc.), blueglasses (meadow grass, bird grass, etc.), fescue (tall fescue, chewings fescue, creeping red fescue, etc.), ryegrasses (darnel, rye grass, etc.), orchard grass, timothy grass, etc.;

Others: flowers (rose, carnation, chrysanthemum, prairie gentian, gypsophila, gerbera, marigold, salvia, petunia, verbena, tulip, aster, gentian, lily, pansy, cyclamen, orchid, convallaria, lavender, stock, ornamental cabbage, primula, poinsettia, gladiolus, cattleya, daisy, cymbidium, begonia, etc.), bio-fuel plants (Jatropha, safflower, camelina, switchgrass, Miscanthus, reed canary grass, giant reed, kenaf, cassava, willow, etc.), ornamental plants, etc.

Among the above plants, corn, rice, soybean, cotton, rapeseed, wheat, etc. are suitable for the present invention.

The “plants” as used herein may be those having resistance, which is imparted by a genetic engineering technique or a cross-breeding method.

The arthropod pest control composition of the present invention can be applied to plants or area in which plants are grown for controlling arthropod pests therein. The plants as used herein include the stems and leaves of plants, the flowers of plants, the fruits of plants, the seeds of plants, and the bulbs of plants. The bulbs as used herein include scaly bulbs, corms, root stalks, tubers, tuberous roots, and rhizophores.

The method for controlling arthropod pests of the present invention comprises applying an effective amount of the arthropod pest control composition of the present invention to plants or area in which plants are grown.

The inventive method also includes embodiments in which the mesoionic compound and the disinfectant compound are applied separately or sequentially.

The “effective amount of the arthropod pest control composition” as used herein means the total amount of the mesoionic compound and the disinfectant compound, which is capable of exerting the controlling effect on an arthropod pest.

Examples of application methods include application to the stems and leaves of plants such as foliage application; application to the seeds of plants; and application to area in which plants are grown such as soil application and submerged application.

Specific examples of the application to the stems and leaves of plants such as foliage application in the present invention include application to the surface of cultivated plants such as ground application by using manual sprayers, power sprayers, boom sprayers or Pancle sprayers, or aerial application or spraying by using radio control helicopters, etc.

Specific examples of the application to the seeds of plants in the present invention include the application of the arthropod pest control composition of the present invention to the seeds or bulbs of plants, more specifically, for example, spray coating treatment on the surface of seeds or bulbs, dressing treatment on the seeds or bulbs of plants, immersion treatment, film coating treatment and pellet coating treatment.

Specific examples of the application to area in which plants are grown such as soil application and submerged application in the present invention include planting hole treatment, plant foot treatment, planting furrow treatment, planting row treatment, broadcast treatment, side row treatment, seedling box treatment, seedbed treatment, mixing with culture soil, mixing with seedbed soil, mixing with a paste fertilizer, water surface treatment, etc.

When the arthropod pest control composition of the present invention is applied to plants or area in which plants are grown, the application amount varies depending on the kinds of plants to be protected, the species or population size of arthropod pests to be controlled, the form of a formulation, the timing of application, weather conditions, etc., but is generally within a range from 0.05 to 10,000 g, preferably from 0.5 to 1,000 g per 1,000 m² of an area where plants are grown, in terms of the total amount of the mesoionic compound and the disinfectant compound.

When the arthropod pest control composition of the present invention is applied to the seeds of plants, the application amount varies depending on the kinds of plants to be protected, the species or population size of arthropod pests to be controlled, the form of a formulation, the timing of application, weather conditions, etc., but is generally within a range from 0.001 to 100 g, preferably from 0.05 to 50 g per 1 kg of the seeds, in terms of the total amount of the mesoionic compound and the disinfectant compound.

The arthropod pest control composition of the present invention is in the form of an emulsifiable concentrate, a wettable powder or a suspension concentrate are generally applied after dilution with water. In this case, the total concentration of the mesoionic compound and the disinfectant compound is generally 0.00001 to 10% by weight, preferably 0.0001 to 5% by weight. The arthropod pest control composition of the present invention in the form of a dust or a granule is generally applied as it is without dilution.

### EXAMPLES

Hereinafter, the present invention will be described in more detail with reference to Formulation Examples and Test Examples, but not limited thereto. In the Examples, the term “part(s)” means part(s) by weight unless otherwise specified, and “the mesoionic compound No. X” (e.g., “the mesoionic compound No. 4”) refers to “compound No. X” (e.g., “compound No. 4”) in Tables 1 to 3.

Formulation Examples will be shown below.

### Formulation Example 1

Twenty parts of the mesoionic compound No. 4, 1 part of a disinfectant compound selected from Group (A) as described below and 35 parts of a mixture (weight ratio 1:1) of white carbon and ammonium polyoxyethylene alkylether sulfate are mixed with water to a total amount of 100 parts, and then the mixture is finely-ground by a wet grinding method to obtain a suspension concentrate.

Group (A): a group consisting of azoxystrobin, fluoxastrobin, pyraclostrobin, the active compound (1), trifloxystrobin, kresoxim-methyl, metominostrobin, picoxystrobin, enestrobin, dimoxystrobin, famoxadone, fenamidone and pyribencarb.

### Formulation Example 2

The procedure as described in Formulation Example 1 is repeated, except that the mesoionic compound No. 5 is employed instead of the mesoionic compound No. 4, to obtain a suspension concentrate.

### Formulation Example 3

The procedure as described in Formulation Example 1 is repeated, except that the mesoionic compound No. 42 is employed instead of the mesoionic compound No. 4, to obtain a suspension concentrate.

### Formulation Example 4

The procedure as described in Formulation Example 1 is repeated, except that the mesoionic compound No. 44 is employed instead of the mesoionic compound No. 4, to obtain a suspension concentrate.

### Formulation Example 5

The procedure as described in Formulation Example 1 is repeated, except that the mesoionic compound No. 1 is employed instead of the mesoionic compound No. 4, to obtain a suspension concentrate.

### Formulation Example 6

Twenty parts of the mesoionic compound No. 4, 1 part of a disinfectant compound selected from Group (A) as described in Formulation Example 1, 1 part of metalaxyl, and 35 parts of a mixture (weight ratio 1:1) of white carbon and ammonium polyoxyethylene alkylether sulfate are mixed with water to a total amount of 100 parts, and then the mixture is finely-ground by a wet grinding method to obtain a suspension concentrate.

### Formulation Example 7

The procedure as described in Formulation Example 6 is repeated, except that the mesoionic compound No. 5 is employed instead of the mesoionic compound No. 4, to obtain a suspension concentrate.

### Formulation Example 8

The procedure as described in Formulation Example 6 is repeated, except that the mesoionic compound No. 42 is employed instead of the mesoionic compound No. 4, to obtain a suspension concentrate.

### Formulation Example 9

The procedure as described in Formulation Example 6 is repeated, except that the mesoionic compound No. 44 is employed instead of the mesoionic compound No. 4, to obtain a suspension concentrate.

### Formulation Example 10

The procedure as described in Formulation Example 6 is repeated, except that the mesoionic compound No. 1 is employed instead of the mesoionic compound No. 4, to obtain a suspension concentrate.

### Formulation Example 11

Twenty parts of the mesoionic compound No. 4, 1 part of a disinfectant compound selected from Group (A) as described in Formulation Example 1, 0.5 parts of metalaxyl M, and 35 parts of a mixture (weight ratio 1:1) of white carbon and ammonium polyoxyethylene alkylether sulfate are mixed with water to a total amount of 100 parts, and then the mixture is finely-ground by a wet grinding method to obtain a suspension concentrate.

### Formulation Example 12

The procedure as described in Formulation Example 11 is repeated, except that the mesoionic compound No. 5 is employed instead of the mesoionic compound No. 4, to obtain a suspension concentrate.

### Formulation Example 13

The procedure as described in Formulation Example 11 is repeated, except that the mesoionic compound No. 42 is employed instead of the mesoionic compound No. 4, to obtain a suspension concentrate.

### Formulation Example 14

The procedure as described in Formulation Example 11 is repeated, except that the mesoionic compound No. 44 is employed instead of the mesoionic compound No. 4, to obtain a suspension concentrate.

### Formulation Example 15

The procedure as described in Formulation Example 11 is repeated, except that the mesoionic compound No. 1 is employed instead of the mesoionic compound No. 4, to obtain a suspension concentrate.

### Formulation Example 16

Ten parts of the mesoionic compound No. 4, 1 part of a disinfectant compound selected from Group (A) as described in Formulation Example 1, 1.5 parts of sorbitan trioleate, and 28 parts of an aqueous solution containing 2 parts of polyvinyl alcohol are mixed, and then the mixture is finely-ground by a wet grinding method. To this mixture, an aqueous solution containing 0.05 parts of xanthane gum and 0.1 parts of magnesium aluminium silicate is added to a total amount of 90 parts, and then 10 parts of propylene glycol is added thereto. The resultant mixture is stirred to obtain a suspension concentrate.

### Formulation Example 17

The procedure as described in Formulation Example 16 is repeated, except that the mesoionic compound No. 5 is employed instead of the mesoionic compound No. 4, to obtain a suspension concentrate.

### Formulation Example 18

The procedure as described in Formulation Example 16 is repeated, except that the mesoionic compound No. 42 is employed instead of the mesoionic compound No. 4, to obtain a suspension concentrate.

### Formulation Example 19

The procedure as described in Formulation Example 16 is repeated, except that the mesoionic compound No. 44 is employed instead of the mesoionic compound No. 4, to obtain a suspension concentrate.

### Formulation Example 20

The procedure as described in Formulation Example 16 is repeated, except that the mesoionic compound No. 1 is employed instead of the mesoionic compound No. 4, to obtain a suspension concentrate.

### Formulation Example 21

Ten parts of the mesoionic compound No. 4, 1 part of a disinfectant compound selected from Group (A) as described in Formulation Example 1, 1 part of metalaxyl, 1.5 parts of sorbitan trioleate, and 28 parts of an aqueous solution containing 2 parts of polyvinyl alcohol are mixed, and then the mixture is finely-ground by a wet grinding method. To this mixture, an aqueous solution containing 0.05 parts of xanthane gum and 0.1 parts of magnesium aluminium silicate is added to a total amount of 90 parts, and then 10 parts of propylene glycol is added thereto. The resultant mixture is stirred to obtain a suspension concentrate.

### Formulation Example 22

The procedure as described in Formulation Example 21 is repeated, except that the mesoionic compound No. 5 is employed instead of the mesoionic compound No. 4, to obtain a suspension concentrate.

### Formulation Example 23

The procedure as described in Formulation Example 21 is repeated, except that the mesoionic compound No. 42 is employed instead of the mesoionic compound No. 4, to obtain a suspension concentrate.

### Formulation Example 24

The procedure as described in Formulation Example 21 is repeated, except that the mesoionic compound No. 44 is employed instead of the mesoionic compound No. 4, to obtain a suspension concentrate.

### Formulation Example 25

The procedure as described in Formulation Example 21 is repeated, except that the mesoionic compound No. 1 is employed instead of the mesoionic compound No. 4, to obtain a suspension concentrate.

### Formulation Example 26

Ten parts of the mesoionic compound No. 4, 1 part of a disinfectant compound selected from Group (A) as described in Formulation Example 1, 0.5 parts of metalaxyl M, 1.5 parts of sorbitan trioleate, and 28 parts of an aqueous solution containing 2 parts of polyvinyl alcohol are mixed, and then the mixture is finely-ground by a wet grinding method. To this mixture, an aqueous solution containing 0.05 parts of xanthane gum and 0.1 parts of magnesium aluminium silicate is added to a total amount of 90 parts, and then 10 parts of propylene glycol is added thereto. The resultant mixture is stirred to obtain a suspension concentrate.

### Formulation Example 27

The procedure as described in Formulation Example 26 is repeated, except that the mesoionic compound No. 5 is employed instead of the mesoionic compound No. 4, to obtain a suspension concentrate.

### Formulation Example 28

The procedure as described in Formulation Example 26 is repeated, except that the mesoionic compound No. 42 is employed instead of the mesoionic compound No. 4, to obtain a suspension concentrate.

### Formulation Example 29

The procedure as described in Formulation Example 26 is repeated, except that the mesoionic compound No. 44 is employed instead of the mesoionic compound No. 4, to obtain a suspension concentrate.

### Formulation Example 30

The procedure as described in Formulation Example 26 is repeated, except that the mesoionic compound No. 1 is employed instead of the mesoionic compound No. 4, to obtain a suspension concentrate.

### Formulation Example 31

Forty parts of the mesoionic compound No. 4, 1 part of a disinfectant compound selected from Group (A) as described in Formulation Example 1, 3 parts of calcium lignin sulfonate, 2 parts of sodium lauryl sulfate, and the rest parts of synthetic hydrated silicon oxide are well mixed while grinding to obtain 100 parts of a wettable powder.

### Formulation Example 32

The procedure as described in Formulation Example 31 is repeated, except that the mesoionic compound No. 5 is employed instead of the mesoionic compound No. 4, to obtain a wettable powder.

### Formulation Example 33

The procedure as described in Formulation Example 31 is repeated, except that the mesoionic compound No. 42 is employed instead of the mesoionic compound No. 4, to obtain a wettable powder.

### Formulation Example 34

The procedure as described in Formulation Example 31 is repeated, except that the mesoionic compound No. 44 is employed instead of the mesoionic compound No. 4, to obtain a wettable powder.

### Formulation Example 35

The procedure as described in Formulation Example 31 is repeated, except that the mesoionic compound No. 1 is employed instead of the mesoionic compound No. 4, to obtain a wettable powder.

### Formulation Example 36

Forty parts of the mesoionic compound No. 4, 1 part of a disinfectant compound selected from Group (A) as described in Formulation Example 1, 1 part of metalaxyl, 3 parts of calcium lignin sulfonate, 2 parts of sodium lauryl sulfate, and the rest parts of synthetic hydrated silicon oxide are well mixed while grinding to obtain 100 parts of a wettable powder.

### Formulation Example 37

The procedure as described in Formulation Example 36 is repeated, except that the mesoionic compound No. 5 is employed instead of the mesoionic compound No. 4, to obtain a wettable powder.

### Formulation Example 38

The procedure as described in Formulation Example 36 is repeated, except that the mesoionic compound No. 42 is employed instead of the mesoionic compound No. 4, to obtain a wettable powder.

### Formulation Example 39

The procedure as described in Formulation Example 36 is repeated, except that the mesoionic compound No. 44 is employed instead of the mesoionic compound No. 4, to obtain a wettable powder.

### Formulation Example 40

The procedure as described in Formulation Example 36 is repeated, except that the mesoionic compound No. 1 is employed instead of the mesoionic compound No. 4, to obtain a wettable powder.

### Formulation Example 41

Forty parts of the mesoionic compound No. 4, 1 part of a disinfectant compound selected from Group (A) as described in Formulation Example 1, 0.5 parts of metalaxyl M, 3 parts of calcium lignin sulfonate, 2 parts of sodium lauryl sulfate, and the rest parts of synthetic hydrated silicon oxide are well mixed while grinding to obtain 100 parts of a wettable powder.

### Formulation Example 42

The procedure as described in Formulation Example 41 is repeated, except that the mesoionic compound No. 5 is employed instead of the mesoionic compound No. 4, to obtain a wettable powder.

### Formulation Example 43

The procedure as described in Formulation Example 41 is repeated, except that the mesoionic compound No. 42 is employed instead of the mesoionic compound No. 4, to obtain a wettable powder.

### Formulation Example 44

The procedure as described in Formulation Example 41 is repeated, except that the mesoionic compound No. 44 is employed instead of the mesoionic compound No. 4, to obtain a wettable powder.

### Formulation Example 45

The procedure as described in Formulation Example 41 is repeated, except that the mesoionic compound No. 1 is employed instead of the mesoionic compound No. 4, to obtain a wettable powder.

The effects of the present invention will be demonstrated below with reference to Test Examples.

### Test Example 1

Each 10 mg of the mesoionic compounds Nos. 4, 5, 42 and 44, azoxystrobin, pyraclostrobin, the active compound (1) and trifloxystrobin was dissolved in 0.2 ml of a 5% (weight/volume) solution of SORGEN TW-20 (manufactured by Dai-ichi Kogyo Seiyaku Co., Ltd.) in acetone (manufactured by Wako Pure Chemical Industries, Ltd.) and then diluted with water to given concentrations.

Each of the water dilutions of the mesoionic compounds Nos. 4, 5, 42 and 44 was mixed with the water dilution of azoxystrobin, pyraclostrobin, the active compound (1) or trifloxystrobin to prepare a test solution.

The seedlings of rice (Oryza sativa; cultivar: Hoshinoyume) at the 1.5 leaf stage were immersed in each test solution. After that, the rice seedlings were air-died and put in plastic test tubes (diameter: 15 mm; height: 100 mm) containing 1 ml of water. Then, 10 third-instar nymphs of Nilaparvata lugens were put in each test tube. The test tubes were placed in a room (25° C., humidity 55%). This is called a treated-section.

In the same manner as in the treated-section, the seedings of rice without any treatment with the test solutions were planted and grown, and then the insects were released. This is called an untreated-section.

Five (5) days after putting them, the tested nymphs were observed for life or death. From the observation results, an insect death rate was calculated by the following Equation 1) and a corrected insect death rate was calculated by the following Equation 2). For each treatment there were 2 replicates. The average values are shown in Tables 4 to 7.

Insect death rate (%)={(Number of tested insects−number of surviving insects)/Number of tested insects}×100 Equation 1);

Corrected insect death rate (%)={(Insect death rate in treated section−Insect death rate in untreated section)/(100−Insect death rate in untreated section)}×100 Equation 2);

TABLE 4

| | Application | Corrected |
|---|---|---|
| | concentration | insect death |
| Tested compounds | [ppm] | rate [%] |
| Mesoionic compound No. 4 + | 10 + 0.1 | 100 |
| Azoxystrobin | | |
| Mesoionic compound No. 4 + | 10 + 10 | 100 |
| Azoxystrobin | | |
| Mesoionic compound No. 4 + | 10 + 0.1 | 100 |
| Pyraclostrobin | | |
| Mesoionic compound No. 4 + | 10 + 10 | 100 |
| Pyraclostrobin | | |
| Mesoionic compound No. 4 + | 10 + 1 | 100 |
| Active compound (1) | | |
| Mesoionic compound No. 4 + | 10 + 1 | 100 |
| Trifloxystrobin | | |

TABLE 5

| | Application | Corrected |
|---|---|---|
| | concentration | insect death |
| Tested compounds | [ppm] | rate [%] |
| Mesoionic compound No. 5 + | 10 + 0.1 | 100 |
| Azoxystrobin | | |
| Mesoionic compound No. 5 + | 10 + 10 | 100 |
| Azoxystrobin | | |
| Mesoionic compound No. 5 + | 10 + 0.1 | 100 |
| Pyraclostrobin | | |
| Mesoionic compound No. 5 + | 10 + 10 | 100 |
| Pyraclostrobin | | |
| Mesoionic compound No. 5 + | 10 + 1 | 100 |
| Active compound (1) | | |
| Mesoionic compound No. 5 + | 10 + 1 | 100 |
| Trifloxystrobin | | |

TABLE 6

| | Application | Corrected |
|---|---|---|
| | concentration | insect death |
| Tested compounds | [ppm] | rate [%] |
| Mesoionic compound No. 42 + | 10 + 0.1 | 100 |
| Azoxystrobin | | |
| Mesoionic compound No. 42 + | 10 + 10 | 100 |
| Azoxystrobin | | |
| Mesoionic compound No. 42 + | 10 + 0.1 | 100 |
| Pyraclostrobin | | |
| Mesoionic compound No. 42 + | 10 + 10 | 100 |
| Pyraclostrobin | | |
| Mesoionic compound No. 42 + | 10 + 1 | 100 |
| Active compound (1) | | |
| Mesoionic compound No. 42 + | 10 + 1 | 100 |
| Trifloxystrobin | | |

TABLE 7

| | Application | Corrected |
|---|---|---|
| | concentration | insect death |
| Tested compounds | [ppm] | rate [%] |
| Mesoionic compound No. 44 + | 10 + 0.1 | 100 |
| Azoxystrobin | | |
| Mesoionic compound No. 44 + | 10 + 10 | 100 |
| Azoxystrobin | | |
| Mesoionic compound No. 44 + | 10 + 0.1 | 100 |
| Pyraclostrobin | | |
| Mesoionic compound No. 44 + | 10 + 10 | 100 |
| Pyraclostrobin | | |
| Mesoionic compound No. 44 + | 10 + 1 | 100 |
| Active compound (1) | | |
| Mesoionic compound No. 44 + | 10 + 1 | 100 |
| Trifloxystrobin | | |

### Test Example 2

Each 10 mg of the mesoionic compounds Nos. 4, 5, 42 and 44, azoxystrobin, pyraclostrobin, the active compound (1), trifloxystrobin and metalaxyl M was dissolved in 0.2 ml of a 5% (weight/volume) solution of SORGEN TW-20 (manufactured by Dai-ichi Kogyo Seiyaku Co., Ltd.) in acetone (manufactured by Wako Pure Chemical Industries, Ltd.) and then diluted with water to given concentrations.

Each of the water dilutions of the mesoionic compounds Nos. 4, 5, 42 and 44 was mixed with the water dilution of azoxystrobin, pyraclostrobin, the active compound (1) or trifloxystrobin, and the water dilution of metalaxyl-M to prepare a test solution.

The seeds of rice (Oryza sativa; cultivar: Hoshinoyume) were treated with each test solution and then planted in plastic cups filled with soil. After 9 days from each treatment, the rice seeds were germinated and 10 third-instar nymphs of Nilaparvata lugens were released onto the rice seedlings. This is called a treated-section.

Specifically, the above treatment was performed as follows: The seeds of rice (Oryza sativa; cultivar: Hoshinoyume) were put in 160-ml plastic cups (diameter: 50 mm, height: 80 mm) and each test solution was added thereto at the rate of 1 ml per 100 seeds. Then, each cup was shaken by hand to apply the test solution to the seeds (dressing treatment). On the same day, 10 treated seeds from each cup were planted in a 160-ml plastic cup (diameter: 50 mm, height: 80 mm) with a lid, filled with a soil, and germinated with occasional sprinkling of water in a climate chamber at 30° C. and 65% relative humidity. After 9 days from each treatment, 10 third-instar nymphs of Nilaparvata lugens were released onto the germinated rice in each cup, and the cup was placed in a room at 25° C. and 55% relative humidity.

In the same manner as in the treated-section, the seeds of rice without any treatment with the test solutions were planted and grown, and then the insects ware released. This is called an untreated-section.

Six(6) days after releasing them, the tested nymphs were observed for life or death. From the observation results, an insect death rate was calculated by the following Equation 3) and a corrected insect death rate was calculated by the following Equation 4). For each treatment there were 2 replicates. The average values are shown in Tables 8 to 11.

Insect death rate (%)={(Number of tested insects−number of surviving insects)/Number of tested insects}×100 Equation 3);

Corrected insect death rate (%)={(Insect death rate in treated section−Insect death rate in untreated section)/(100−Insect death rate in untreated section)}×100 Equation 4);

TABLE 8

| | Application | Corrected |
|---|---|---|
| | amount | insect death |
| Tested compounds | [mg ai/seed] | rate [%] |
| Mesoionic compound No. 4 + | 0.05 + | 100 |
| Azoxystrobin + | 0.0005 + | |
| Metalaxyl M | 0.0005 | |
| Mesoionic compound No. 4 + | 0.05 + | 100 |
| Azoxystrobin + | 0.05 + | |
| Metalaxyl M | 0.05 | |
| Mesoionic compound No. 4 + | 0.05 + | 100 |
| Pyraclostrobin + | 0.0005 + | |
| Metalaxyl M | 0.0005 | |
| Mesoionic compound No. 4 + | 0.05 + | 100 |
| Pyraclostrobin + | 0.05 + | |
| Metalaxyl M | 0.05 | |
| Mesoionic compound No. 4 + | 0.05 + | 100 |
| Active compound (1) + | 0.005 + | |
| Metalaxyl M | 0.005 | |
| Mesoionic compound No. 4 + | 0.05 + | 100 |
| Trifloxystrobin + | 0.005 + | |
| Metalaxyl M | 0.005 | |

TABLE 9

| | Application | Corrected |
|---|---|---|
| | amount | insect death |
| Tested compounds | [mg ai/seed] | rate [%] |
| Mesoionic compound No. 5 + | 0.05 + | 100 |
| Azoxystrobin + | 0.0005 + | |
| Metalaxyl M | 0.0005 | |
| Mesoionic compound No. 5 + | 0.05 + | 100 |
| Azoxystrobin + | 0.05 + | |
| Metalaxyl M | 0.05 | |
| Mesoionic compound No. 5 + | 0.05 + | 100 |
| Pyraclostrobin + | 0.0005 + | |
| Metalaxyl M | 0.0005 | |
| Mesoionic compound No. 5 + | 0.05 + | 100 |
| Pyraclostrobin + | 0.05 + | |
| Metalaxyl M | 0.05 | |
| Mesoionic compound No. 5 + | 0.05 + | 100 |
| Active compound (1) + | 0.005 + | |
| Metalaxyl M | 0.005 | |
| Mesoionic compound No. 5 + | 0.05 + | 100 |
| Trifloxystrobin + | 0.005 + | |
| Metalaxyl M | 0.005 | |

TABLE 10

| | Application | Corrected |
|---|---|---|
| | amount | insect death |
| Tested compounds | [mg ai/seed] | rate [%] |
| Mesoionic compound No. 42 + | 0.05 + | 100 |
| Azoxystrobin + | 0.0005 + | |
| Metalaxyl M | 0.0005 | |
| Mesoionic compound No. 42 + | 0.05 + | 100 |
| Azoxystrobin + | 0.05 + | |
| Metalaxyl M | 0.05 | |
| Mesoionic compound No. 42 + | 0.05 + | 100 |
| Pyraclostrobin + | 0.0005 + | |
| Metalaxyl M | 0.0005 | |
| Mesoionic compound No. 42 + | 0.05 + | 100 |
| Pyraclostrobin + | 0.05 + | |
| Metalaxyl M | 0.05 | |
| Mesoionic compound No. 42 + | 0.05 + | 100 |
| Active compound (1) + | 0.005 + | |
| Metalaxyl M | 0.005 | |
| Mesoionic compound No. 42 + | 0.05 + | 100 |
| Trifloxystrobin + | 0.005 + | |
| Metalaxyl M | 0.005 | |

TABLE 11

| | Application | Corrected |
|---|---|---|
| | amount | insect death |
| Tested compounds | [mg ai/seed] | rate [%] |
| Mesoionic compound No. 44 + | 0.05 + | 100 |
| Azoxystrobin + | 0.0005 + | |
| Metalaxyl M | 0.0005 | |
| Mesoionic compound No. 44 + | 0.05 + | 100 |
| Azoxystrobin + | 0.05 + | |
| Metalaxyl M | 0.05 | |
| Mesoionic compound No. 44 + | 0.05 + | 100 |
| Pyraclostrobin + | 0.0005 + | |
| Metalaxyl M | 0.0005 | |
| Mesoionic compound No. 44 + | 0.05 + | 100 |
| Pyraclostrobin + | 0.05 + | |
| Metalaxyl M | 0.05 | |
| Mesoionic compound No. 44 + | 0.05 + | 100 |
| Active compound (1) + | 0.005 + | |
| Metalaxyl M | 0.005 | |
| Mesoionic compound No. 44 + | 0.05 + | 100 |
| trifloxystrobin + | 0.005 + | |
| Metalaxyl M | 0.005 | |

In Tables 8 to 11, the term “mg ai/seed” means milligram compound per one seed applied in the test.

### Test Example 3

Each 10 mg of the mesoionic compounds No. 1 and azoxystrobin was dissolved in 0.2 ml of a 5% (weight/volume) solution of SORGEN TW-20 (manufactured by Dai-ichi Kogyo Seiyaku Co., Ltd.) in acetone (manufactured by Wako Pure Chemical Industries, Ltd.) and then diluted with water to given concentrations.

Each of the water dilution of the mesoionic compound No. 1 was mixed with the water dilution of azoxystrobin to prepare a test solution.

The seeds of rice (Oryza sativa; cultivar: Hoshinoyume) were treated with each test solution and then planted in plastic cups filled with soil. After 10 days from each treatment, the rice seeds were germinated and 10 third-instar nymphs of Nilaparvata lugens were released onto the rice seedlings. This is called a treated-section.

Specifically, the above treatment was performed as follows: The seeds of rice (Oryza sativa; cultivar: Hoshinoyume) were put in 160-ml plastic cups (diameter: 50 mm, height: 80 mm) and each test solution was added thereto at the rate of 1 ml per 100 seeds. Then, each cup was shaken by hand to apply the test solution to the seeds (dressing treatment). On the same day, 10 treated seeds from each cup were planted in a 160-ml plastic cup (diameter: 50 mm, height: 80 mm) with a lid, filled with a soil, and germinated with occasional sprinkling of water in a climate chamber at 30° C. and 65% relative humidity. After 10 days from each treatment, 10 third-instar nymphs of Nilaparvata lugens were released onto the germinated rice in each cup, and the cup was placed in a room at 25° C. and 55% relative humidity.

In the same manner as in the treated-section, the seeds of rice without any treatment with the test solutions were planted and grown, and then the insects were released. This is called an untreated-section.

Six(6) days after releasing them, the tested nymphs were observed for life or death. From the observation results, an insect death rate was calculated by the following Equation 4) and a corrected insect death rate was calculated by the following Equation 5). For each treatment there were 2 replicates. The average values are shown in Table 12.

Insect death rate (%)={(Number of tested insects−number of surviving insects)/Number of tested insects}×100 Equation 4);

Corrected insect death rate (%)={(Insect death rate in treated section−Insect death rate in untreated section)/(100−Insect death rate in untreated section)}×100 Equation 5);

TABLE 12

| | Application | Corrected |
|---|---|---|
| | amount | insect death |
| Tested compounds | [mg ai/seed] | rate [%] |
| Mesoionic compound No. 1 + | 0.05 + | 100 |
| azoxystrobin | 0.0005 | |
| Mesoionic compound No. 1 + | 0.05 + | 100 |
| azoxystrobin | 0.05 | |

In Table 12, the term “mg ai/seed” means milligram compound per one seed applied in the test.

### Test Example 4

Each 10 mg of the mesoionic compounds Nos. 1, 4, 5, 42 and 44, and fluoxastrobin is dissolved in 0.2 ml of a 5% (weight/volume) solution of SORGEN TW-20 (manufactured by Dai-ichi Kogyo Seiyaku Co., Ltd.) in acetone (manufactured by Wako Pure Chemical Industries, Ltd.) and then diluted with water to given concentrations.

Each of the water dilutions of the mesoionic compounds Nos. 1, 4, 5, 42 and 44 is mixed with the water dilution of fluoxastrobin to prepare a test solution.

One soybean seed is spray-coated in a 15 ml centrifuge tube with each test solution (5 μl) containing 2 mg of the mesoionic compound Nos. 1, 4, 5, 42 or 44, and 0.2 mg of fluoxastrobin, and then the treated seed is planted in a 1/10,000a Wagner pot filled with soil and grown in a greenhouse for 12 days. About 20 insects of Aulacorthum solani are released into each pot. This is called a treated-section.

In the same manner as in the treated-section, one soybean seed without any treatment with the test solutions is planted and grown, and then the insects are released. This is called an untreated-section.

Six(6) days after releasing them, the number of Aulacorthum solani is counted in the treated-section and the untreated-section, and a controlling value is determined by the following equation. An insect death rate is calculated according to the following equation.

Controlling value (%)={1−(Cb×Tai)/(Cai×Tb)}×100

wherein,

Cb: the number of insects in an untreated section before treatment

Cai: the number of insects in an untreated section on observation

Tb: the number of insects in a treated-section before treatment

Tai: the number of insects in a treated section on observation

As a result, it is found that the controlling effect on arthropod pests obtained in a treated section is better than that obtained in an untreated section.

1. An arthropod pest control composition comprising a compound represented by formula (I): 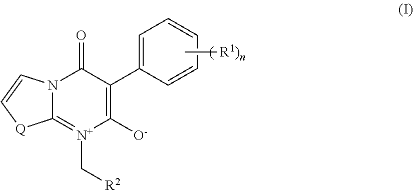
wherein,
Q represents CR⁵═CR⁶, S, O or NCH₃,
R¹ represents a halogen atom, cyano, nitro, an optionally halogenated C1-C4 alkyl group,
an optionally halogenated C2-C4 alkenyl group, an optionally halogenated C2-C4 alkynyl group or an optionally halogenated C1-C4 alkoxy group,
n represents an integer of 0 to 3,
R² represents the following R^{2a}, R^{2b}, R^{2c} or R^{2d}; 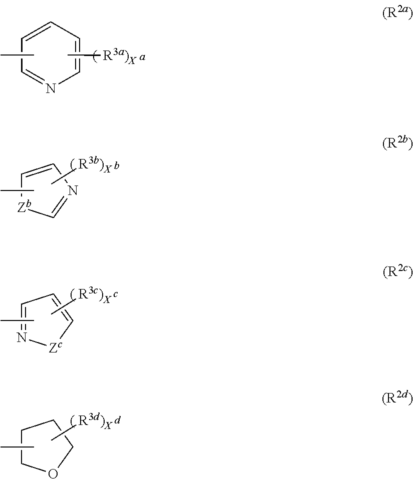
wherein,
R^{3a}, R^{3b} and R^{3c} each independently represent a halogen atom, cyano, nitro, an optionally halogenated C1-C4 alkyl group, an optionally halogenated C2-C4 alkynyl group or an optionally halogenated C1-C4 alkoxy group,
R^{3d} represents a halogen atom, cyano, nitro, an optionally halogenated C1-C4 alkyl group or an optionally halogenated C1-C4 alkoxy group,
X^{a}, X^{b}, X^{c} and X^{d} each independently represent 0, 1 or 2,
Z^{b} and Z^{c} each independently represent O, S or NR⁷,
R⁷ represents a hydrogen atom or an optionally halogenated C1-C4 alkyl group,
wherein,
when X^{a} represents 2, then two R^{3a}'s may be the same or different,
when X^{b} represents 2, then two R^{3b}'s may be the same or different,
when X^{c} represents 2, then two R^{3c}'s may be the same or different, and
when X^{d} represents 2, then two R^{3d}'s may be the same or different,
R⁵ represents a hydrogen atom or a fluorine atom, and
R⁶ represents a hydrogen atom, a fluorine atom, a difluoromethyl group or a trifluoromethyl group,
wherein,
when n represents 2 or 3, then plural R¹'s may be the same or different; and
at least one disinfectant compound selected from Group (A);
Group (A): a group consisting of azoxystrobin, fluoxastrobin, pyraclostrobin, a compound represented by the following formula (I), trifloxystrobin, kresoxim-methyl, metominostrobin, picoxystrobin, enestrobin, dimoxystrobin, famoxadone, fenamidone and pyribencarb;

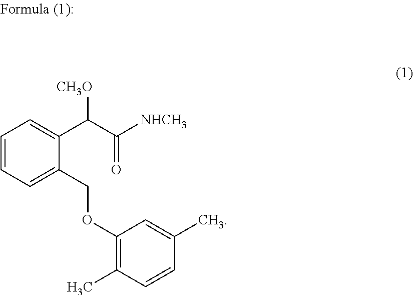

2. The arthropod pest control composition according to claim 1, wherein the weight ratio of the compound represented by formula (I) to the disinfectant compound 10,000:1 to 0.01:1.

3. The arthropod pest control composition according to claim 1, wherein the disinfectant compound is azoxystrobin, pyraclostrobin, a compound represented by the following formula (1) or trifloxystrobin;

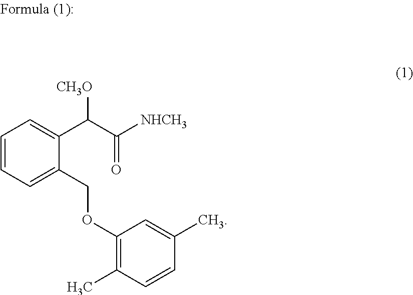

4. The arthropod pest control composition according to claim 1, wherein the composition further comprises metalaxyl or metalaxyl M.

5. The arthropod pest control composition according to claim 4, wherein the weight ratio of the compound represented by formula (I) to metalaxyl or metalaxyl M is 10,000:1 to 0.01:1.

6. A method for controlling arthropod pests, which comprises applying an effective amount of the arthropod pest control composition according to claim 1 to plants or area in which plants are grown.

7. The method for controlling arthropod pests according to claim 6, wherein the plants or area in which plants are grown is the seeds of plants.
